(19) United States
(12) Patent Application Publication
(10) Pub. No.: US 2010/0220292 A1
(43) Pub. Date: Sep. 2, 2010

(54) VISION MEASURING SYSTEM AND ASSISTANT FOCUSING SYSTEM THEREOF

(75) Inventors: CHIH-KUANG CHANG, Tu-Cheng (TW); HENG ZHANG, Shenzhen City (CN)

Correspondence Address: PCE INDUSTRY, INC.;ATT. Steven Reiss, 288 SOUTH MAYO AVENUE, CITY OF INDUSTRY, CA, 91789, US

(73) Assignees: HONG FU JIN PRECISION INDUSTRY (ShenZhen) CO., LTD., Shenzhen City (CN); HON HAI PRECISION INDUSTRY CO., LTD., Tu-Cheng (TW)

(21) Appl. No.: 12/421,585

(22) Filed: Apr. 9, 2009

(30) Foreign Application Priority Data
Mar. 2, 2009 (CN) 200910300632.9

(51) Int. Cl. A61B 3/10

(52) U.S. Cl. 351/221

(57) ABSTRACT

A vision measuring system includes an optical system, an image capture apparatus for receiving an image of the object through the optical system and converting the image to electronic signals, a first illuminator for emitting light to the object, and an assistant focusing system. The optical system includes a first object lens, a second object lens, and a partial reflector located between the first and second object lens. The partial reflector is unparallel to the second object lens. The assistant focusing system includes a second illuminator and a pattern slide. Patterns are attached in checkerboard fashion on the pattern slide. Light from the second illuminator is able to pass through the pattern slide, the partial reflector, and the second object lens to make the patterns of the pattern slide be projected onto the surface of the object.

### CROSS-REFERENCE TO RELATED APPLICATION

Relevant subject matter is disclosed in a co-pending U.S. patent application (Attorney Docket No. US25394) filed on the same date and having a title of “FOCUS ASSISTING DEVICE”, which is assigned to the same assignee as this patent application.

### BACKGROUND

1. Technical Field

Embodiments of the present disclosure relate to measuring systems and, more particularly, to a vision measuring system and an assistant focusing system of the vision measuring system.

2. Description of Related Art

Auto-focus systems are provided in optical measuring instruments such as what are used in vision measuring systems. A contrast method to focus lenses based on the contrast of an image of an object surface to be measured is well known in the auto-focus system field. However, the image is first taken by a charge coupled device (CCD) camera, it is difficult for the contrast method to focus precisely on an essentially low contrast surface such as a mirror-finish surface or a glass surface.

### BRIEF DESCRIPTION OF THE DRAWINGS

FIG. 1 is an isometric view of an embodiment of a vision measuring system, the vision measuring system includes an assistant focusing system, and a pattern slide.

FIG. 2 is an isometric view of the assistant focusing system of FIG. 1.

FIG. 3 is a cross-sectional view of FIG. 2.

FIG. 4 is a schematic diagram of the pattern slide of FIG. 1.

FIG. 5 is a block diagram of the vision measuring system of FIG. 1.

### DETAILED DESCRIPTION

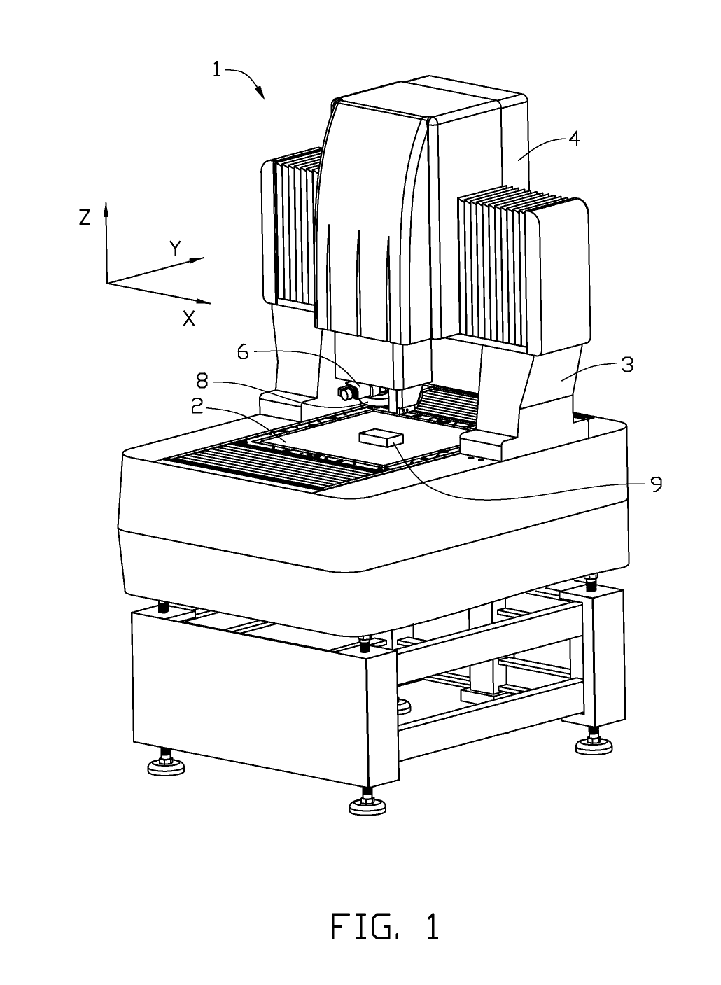

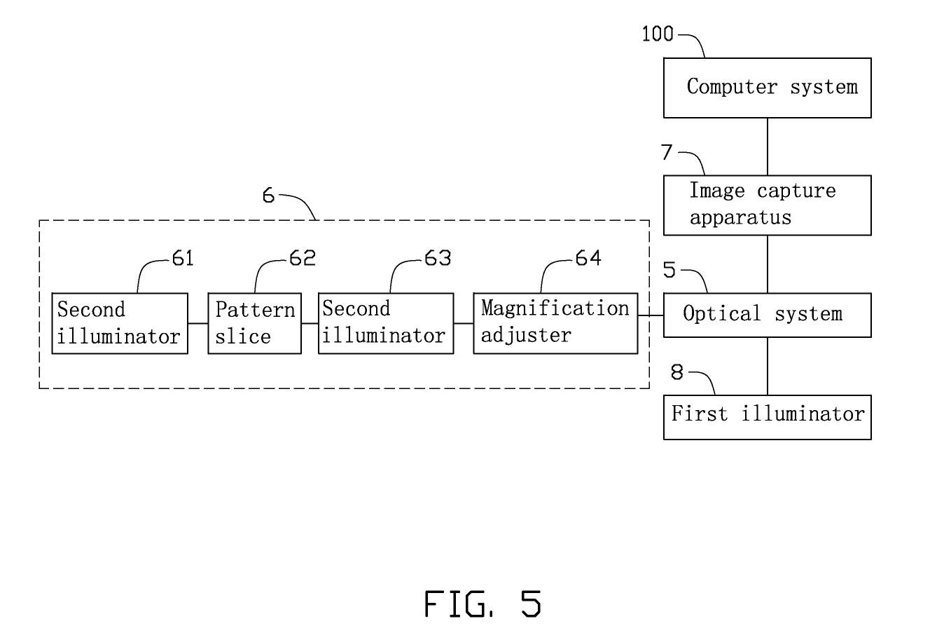

Referring to FIGS. 1 and 5, an embodiment of a vision measuring system 1 includes a horizontal worktable 2, a bracket 3, a top cover 4, an optical system 5, an assistant focusing system 6, an image capture apparatus 7, and a first illuminator 8. The bracket 3 is mounted to the worktable 2. The top cover 4 is mounted to a middle part of the bracket 3. The optical system 5 and the image capture apparatus 7 are coupled to the top cover 4 and positioned such that the image capture apparatus 7 captures an image of an object 9 positioned on the worktable 2 to be measured via the optical system 5. In one embodiment, the image capture apparatus 7 may be a charge coupled device (CCD) camera.

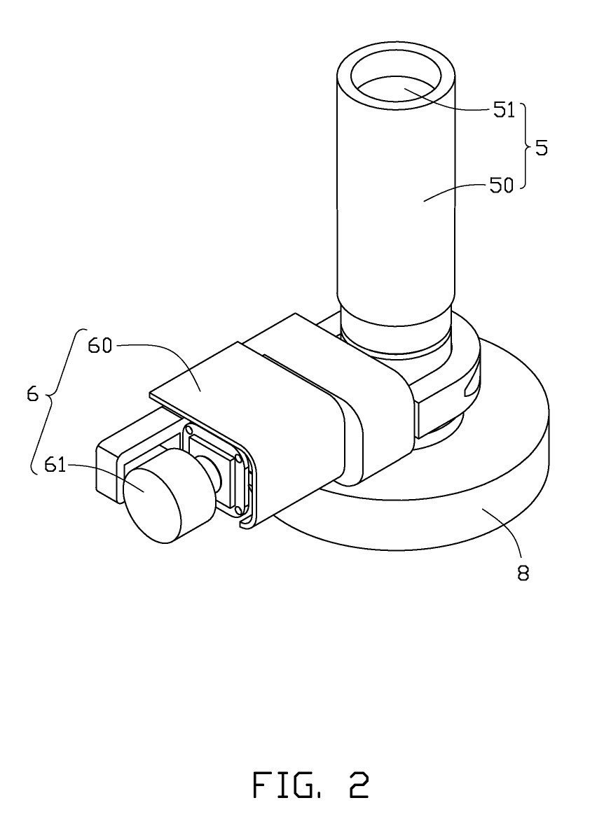

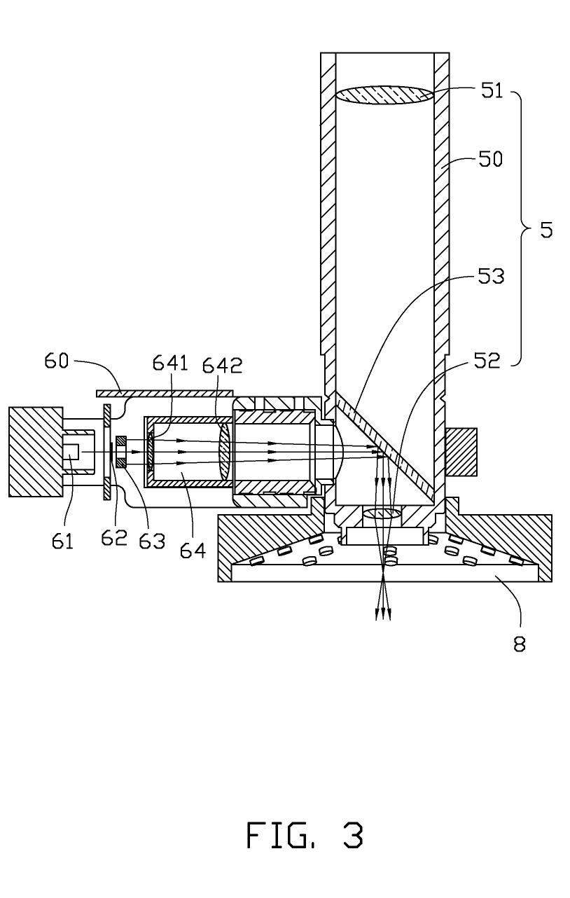

Referring to FIGS. 2 and 3, the optical system 5 includes a cylinder-shaped first shell 50, first and second object lenses 51 and 52 mounted to first and second ends of the first shell 50, and a partial reflector 53. The image capture apparatus 7 is mounted to the first end of the first shell 50, opposite to the second object lens 52 relative to the first object lens 51, and arranged along an optical axis of the optical system 5. The first illuminator 8 is mounted to the second end of the first shell 50, opposite to the first object lens 51 relative to the second object lens 52, and configured to emit light to the object 9. The image capture apparatus 7 is configured to receive an image of the object 9 through the optical system 5.

The partial reflector 53 is located between the first object lens 51 and the second object lens 52 in the first shell 50. There is an angle of about 450 between the optical axis of the optical system 5 and the partial reflector 53.

The assistant focusing system 6 is perpendicularly mounted to a circumference of an end of the optical system 5, and configured to project patterns on a surface of the object 9. The assistant focusing system 6 includes a second shell 60, a second illuminator 61, a pattern slide 62, a third illuminator 63, and a magnification adjuster 64. The second illuminator 61 is located at a distal end of the second shell 60, opposite to the optical system 5. The second illuminator 61, the pattern slide 62, the third illuminator 63, and the magnification adjuster 64 are located in the second shell 60 in that order.

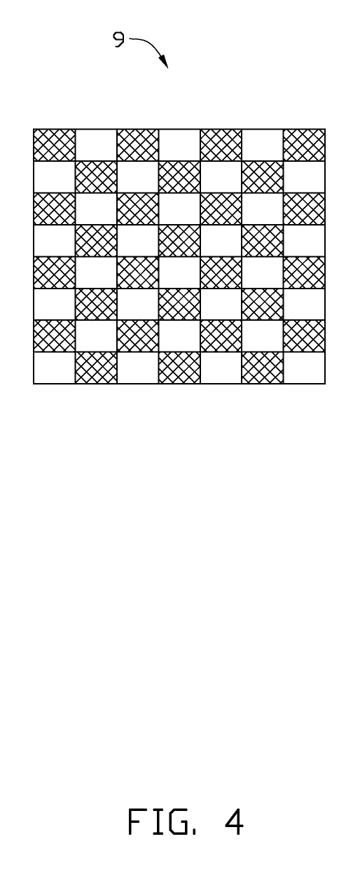

Referring to FIG. 4, the pattern slide 62 is a transparent element, such as is normally used for a glass slide. Patterns, such as a plurality of square portions which are transparent and nontransparent are attached in checkerboard fashion on a surface of the pattern slide 62 as shown in FIG. 4. The magnification adjuster 64 includes a third object lens 641 and a fourth object lens 642. A distance between the third and fourth object lenses 641 and 642 can be adjusted to change a size of the projected patterns on the surface of the object 9. In other embodiments, the patterns of the pattern slide 62 can be formed in some other pattern or image.

In use, if the object 9 has smooth low-contrast surfaces, the second illuminator 61 and the third illuminator 63 are turned on. Light from the second illuminator 61 passes through the pattern slide 62, the third object lens 641, the fourth object lens 642, the semi-transparent reflector 53, and the second object lens 52 to be projected onto the surface of the object 9. As a result, the patterns of the surface of the pattern slide 62 are projected onto the surface of the object 9. In addition, light from the third illuminator 63 passes through the semi-transparent reflector 53 and the second object lens 52 to be projected onto the surface of the object 9, to illuminate the object 9. The optical system 5 focuses light reflected by the surface of the object 9 on an image capturing surface of the image capture apparatus 7, which captures an image of the surface of the object 9 and converts the image into electronic signals, to transmit the electronic signals to a computer system 100. The computer system 100 computes a focus setting for precisely focusing on the object 9 according to the contrast of the image of the object 9 using any one or more methods known in the art.

If the object 9 has smooth high-contrast surfaces, the second illuminator 61 is turned off. The third illuminator 63 and the first illuminator 8 are turned on. Light from the third illuminator 63 passes through the semi-transparent reflector 53 and the second object lens 52 to be projected onto the surface of the object 9, to illuminate the object 9. Light from the first illuminator 8 is projected onto a region to be measured on the surface of the object 9. The image capture apparatus 7 receives an image of the object 9 through the optical system 5, converts the image to electronic signals, and then transmits the electronic signals to the computer system 100. The computer system 100 computes a focus setting for precisely focusing on the object 9, according to the contrast of the image of the object 9, in a well known manner.

The foregoing description of the exemplary embodiments of the disclosure has been presented only for the purposes of illustration and description and is not intended to be exhaustive or to limit the disclosure to the precise forms disclosed. Many modifications and variations are possible in lights of the above everything. The embodiments were chosen and described in order to explain the principles of the disclosure and their practical application so as to enable others of ordinary skill in the art to utilize the disclosure and various embodiments and with various modifications as are suited to the particular use contemplated. Alternative embodiments will become apparent to those of ordinary skills in the art to which the present disclosure pertains without departing from its spirit and scope. Accordingly, the scope of the present disclosure is defined by the appended claims rather than the foregoing description and the exemplary embodiments described therein.

What is claimed is:

1. A vision measuring system comprising:
an optical system comprising a first object lens, a second object lens, and a partial reflector located between the first and second object lens, wherein the partial reflector is unparallel to the second object lens;
an image capture apparatus for receiving an image of the object through the optical system, and converting the image to electronic signals;
a first illuminator for emitting light to the object; and
an assistant focusing system comprising a second illuminator and a pattern slide, wherein patterns are attached on a surface of the pattern slide, wherein light from the second illuminator is capable of passing through the pattern slide, the partial reflector, and the second object lens to make patterns of the surface of the pattern slide be projected onto the surface of the object.

2. The vision measuring system of claim 1, wherein the assistant focusing system further comprises a third illuminator, the third illuminator is located adjacent to the pattern slide and opposite to the second illuminator relative to the pattern slide, light from the third illuminator is capable of passing through the partial reflector and the second object lens to be projected onto the surface of the object.

3. The vision measuring system of claim 2, wherein the assistant focusing system further comprises a magnification adjuster, the magnification adjuster is located adjacent to the third illuminator and opposite to the pattern slide relative to the third illuminator, wherein the magnification adjuster comprises a third object lens and a fourth object lens, and a distance between the third and fourth object lens is adjustable.

4. The vision measuring system of claim 1, wherein a plurality of square transparent portions and a plurality of square nontransparent portions are attached in checkerboard fashion on the pattern slide.

5. The vision measuring system of claim 2, wherein a plurality of square transparent portions and a plurality of square nontransparent portions are attached in checkerboard fashion on the pattern slide.

6. The vision measuring system of claim 3, wherein a plurality of square transparent portions and a plurality of square nontransparent portions are attached in checkerboard fashion on the pattern slide.

7. An assistant focusing system comprising:
a first illuminator to emit light; and
a pattern slide arranged in that order with the first illuminator, wherein patterns are attached on the pattern slide;
wherein the light from the first illuminator is capable of passing through the pattern slide to make patterns of the surface of the pattern slide be projected onto an object.

8. The assistant focusing system of claim 7, further comprising a second illuminator, wherein the second illuminator is located adjacent to the pattern slide, opposite to the first illuminator.

9. The assistant focusing system of claim 8, further comprising a magnification adjuster, wherein the magnification adjuster is located adjacent to the second illuminator, opposite to the pattern slide, wherein the magnification adjuster comprises a first object lens and a second object lens, and a distance between the first and second object lens is adjustable.

10. The assistant focusing system of claim 7, wherein a plurality of square transparent portions and a plurality of square nontransparent portions are attached in checkerboard fashion on the pattern slide.

11. The assistant focusing system of claim 8, wherein a plurality of square transparent portions and a plurality of square nontransparent portions are attached in checkerboard fashion on the pattern slide.

12. The assistant focusing system of claim 9, wherein a plurality of square transparent portions and a plurality of square nontransparent portions are attached in checkerboard fashion on the pattern slide.
